(19) United States
(12) Patent Application Publication
(10) Pub. No.: US 2024/0226083 A9
(43) Pub. Date: Jul. 11, 2024

(54) MEDICAMENT FOR TREATMENT AND/OR PROPHYLACTIC TREATMENT OF CANCERS

(71) Applicant: Belina Pharma AB, Lund (SE)

(72) Inventor: Håkan OLSSON, Lund (SE)

(21) Appl. No.: 17/769,502

(22) PCT Filed: Oct. 14, 2020
(86) PCT No.: PCT/SE2020/050978

§ 371 Date: Apr. 15, 2022

(30) Foreign Application Priority Data
Oct. 18, 2019 (SE) 1951181-5

(51) Int. Cl. A61K 31/4545; A61K 45/06; A61P 35/00

(52) U.S. Cl. CPC A61K 31/4545; A61K 45/06; A61P 35/00

(57) ABSTRACT

Disclosed herein is desloratadine for use in the treatment and/or prophylactic treatment of lung cancer, prostate cancer, pancreatic cancer, gastric cancer, kidney cancer, ovarian cancer, colorectal cancer, Hodgins's disease, bladder cancer, liver cancer, and/or nasopharyngeal cancer.

### FIELD OF THE INVENTION

This invention pertains in general to the field of treatment of cancers. More particularly the invention relates to a medicament for treatment and prophylactic treatment of lung cancer, prostate cancer, pancreatic cancer, gastric cancer, kidney cancer, ovarian cancer, colorectal cancer, Hodgins's disease, bladder cancer, liver cancer, and/or nasopharyngeal cancer.

### BACKGROUND OF THE INVENTION

Although cancer treatment has progressed enormously the last decades, there are still several cancers which are both inoperable and where alternative treatments cannot offer high survivability. One such cancer is pancreatic cancer, where the 5 year survival is still below 10%. Cancers may also be radio- and chemotherapy resistant, whereby the success rate for conventional cancer treatment may be low. However, such cancers may be susceptible to other treatments.

These circumstances have led to a recent surge in research into alternative therapies, such as therapies aiming to boost anti-tumour immune responses in cancer patients. However, not all patients respond to the new treatments and recurrence of cancers still present a problem.

As such, there is still a need to develop new treatments and preventive measures for cancer treatment.

### SUMMARY OF THE INVENTION

It is an object of the present invention, considering the disadvantages mentioned above, to provide desloratadine for use in the treatment and/or prophylactic treatment of cancers, specifically lung cancer, prostate cancer, pancreatic cancer, gastric cancer, kidney cancer, ovarian cancer, colorectal cancer, Hodgins's disease, bladder cancer, liver cancer, and/or nasopharyngeal cancer.

### BRIEF DESCRIPTION OF THE DRAWINGS

These and other aspects, features and advantages of which the invention is capable of will be apparent and elucidated from the following description of embodiments of the present invention, reference being made to the accompanying drawings, in which

FIG. 1 is a Forest plot showing cancer specific survival in patients taking and not taking desloratadine,

FIG. 2 is a Forest plot showing cancer specific survival in patients taking and not taking clemastine,

FIG. 3 is a Forest plot showing cancer specific survival in patients taking and not taking loratadin,

FIG. 4 is a Forest plot showing cancer specific survival in patients taking and not taking ceterizine,

FIG. 5 is a Forest plot showing cancer specific survival in patients taking and not taking ebastine,

FIG. 6 is a Forest plot showing cancer specific survival in patients taking and not taking fexofenadine,

FIG. 7 is a survival plot showing cancer specific survival in patients with prostate cancer, taking and not taking desloratadine,

FIG. 8 is a survival plot showing cancer specific survival in patients with gastric cancer, taking and not taking desloratadine,

FIG. 9 is a survival plot showing cancer specific survival in patients with ovarian cancer, taking and not taking desloratadine,

FIG. 10 is a survival plot showing cancer specific survival in patients with kidney cancer, taking and not taking desloratadine,

FIG. 11 is a survival plot showing cancer specific survival in patients with uterus cancer, taking and not taking desloratadine,

FIG. 12 is a survival plot showing cancer specific survival in patients with colo-rectal cancer, taking and not taking desloratadine,

FIG. 13 is a survival plot showing cancer specific survival in patients with pancreatic cancer, taking and not taking desloratadine,

FIG. 14 is a survival plot showing cancer specific survival in patients with lung cancer, taking and not taking desloratadine,

FIG. 15 is a survival plot showing cancer specific survival in patients with brain cancer, taking and not taking desloratadine,

FIG. 16 is a survival plot showing cancer specific survival in patients with non-Hodgkin's lymphoma, taking and not taking desloratadine,

FIG. 17 is a survival plot showing cancer specific survival in patients with Hodgkin's disease, taking and not taking desloratadine, and

FIG. 18 is a survival plot showing cancer specific survival in patients with Acute leukemia, taking and not taking desloratadine.

### DESCRIPTION OF EMBODIMENTS

The following description focuses on an embodiment of the present invention applicable to a pharmaceutical composition for use in the treatment and prophylactic treatment of cancers.

Cancers may be inoperable due to their type, location, or because there are multiple tumors. One such example is advanced pancreatic cancer. For such cancers, other treatments are required to help control the cancer. It is common to be offered chemotherapy, and sometimes chemoradiotherapy, to shrink the cancer and slow down its growth. For cancers which are found to be found both highly radio- and chemotherapy resistant, the success rate for conventional cancer treatment may be low. Sadly, for several of these cancers, mortality rates are also very high. Thus, there is a need for new treatments that shows effect on such cancers.

Table 1 summarizes the 5 year survival rates for Swedish cancer patients diagnosed with the listed cancers as of 2016 (National Board of Health, Sweden). Although several cancers are treatable to a high extent, there are still several cancers with a 5 year survival rate below 30%.

TABLE 1
Cancer survival in Sweden 2016

| | 5 year | number |
|---|---|---|
| Cancer | survival | of cases |
| Acute leukemia | 35% | 640 |
| Non-Hodgkin's lymphoma | 75% | 841 |
| Brain cancer | 60% | 1353 |
| Uterine cancer | 84% | 1373 |
| Ear-nose-throat | 75% | 382 |
| Bladder cancer | 72% | 3156 |
| Breast cancer | 92% | 8923 |
| Pancreatic cancer | 8% | 1217 |
| Melanoma | 92% | 4151 |
| Lung cancer | 20% | 3938 |
| Gastric cancer | 25% | 796 |
| Kidney cancer | 75% | 1267 |
| Prostate cancer | 93% | 10474 |
| Colon cancer | 65% | 4702 |
| Rectal cancer | 66% | 2153 |
| Ovarian cancer | 55% | 544 |
| Hodgkin's disease | 86% | 183 |

One such kind of novel treatment is immunogenic treatment, therapies aiming to boost anti-tumor immune responses in cancer patients, such as check-point therapy. Our immune system has a set of brakes—or checkpoints—that stop it from killing healthy cells. Sometimes, cancer cells take advantage of this and hide from your defense, almost disguising themselves as normal cells. That way, your system doesn't see the cancer cell as an invader. These drugs help your immune system see the cancer as a problem, and fight it. Checkpoint inhibitors are in clinical trials for many different cancers, and currently trials are made for patients with cancers, such as metastatic melanoma, lung, bladder, kidney, head and neck cancer and Hodgkin's lymphoma.

Among these immunotherapies, neutralizing antibodies targeting the immune checkpoints T-lymphocyte-associated protein 4 (CTLA-4) and programmed cell death protein 1 (PD-1) are being hailed as particularly successful. These antibodies have resulted in dramatic improvements in disease outcome and are now clinically approved in many countries. However, the majority of advanced stage patients do not respond or will relapse, and the hunt for the “magic bullet” to treat the disease continues.

Database mining has proven a very powerful tool for finding unseen trends in treatment results. The Swedish Drug Prescription Registry contains data on pharmaceuticals, supplies, and food taken prescription or equivalent in pharmacy from 2005 onwards. The number of prescriptions is almost 100 million a year. The register is updated with new information every month. The Swedish Cancer Registry was founded in 1958 and covers the entire population of Sweden. Approximately 50 000 malignant cases of cancer is registered every year in Sweden. It is compulsory for every health care provider to report newly detected cancer cases to the registry. A report has to be sent for every cancer case diagnosed at clinical-, morphological-, other laboratory examinations as well as cases diagnosed at autopsy. Thus, these registers provide a very good foundation for database mining seen from an international standard.

In WO 2016/116438, whilst looking for treatment relating to survival improvement of breast cancer, it was found that treatment using H1 receptor antagonists, especially desloratadine or ebastin, has a positive effect on breast cancer survival. No general effect on cancer was found at the time of the study, only the specific effect on breast cancer.

It has now been investigated the effects of the six major H1-antihistamines (cetirizine, clemastine, desloratadine, ebastine, fexofenadine and loratadine) used in Sweden on cancer-specific and overall mortality in a nation-wide register-based study. Both peri- and post-diagnostic antihistamine use was analyzed, using Cox regression models, as well as different subgroups of patients with regard to age, gender (as a proxy for sex) and tumor status.

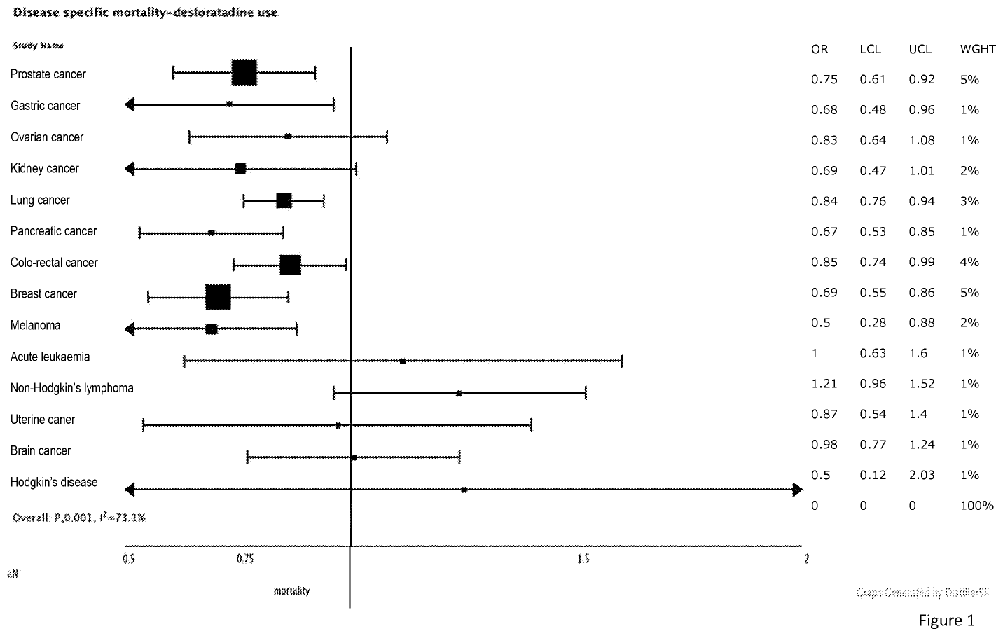

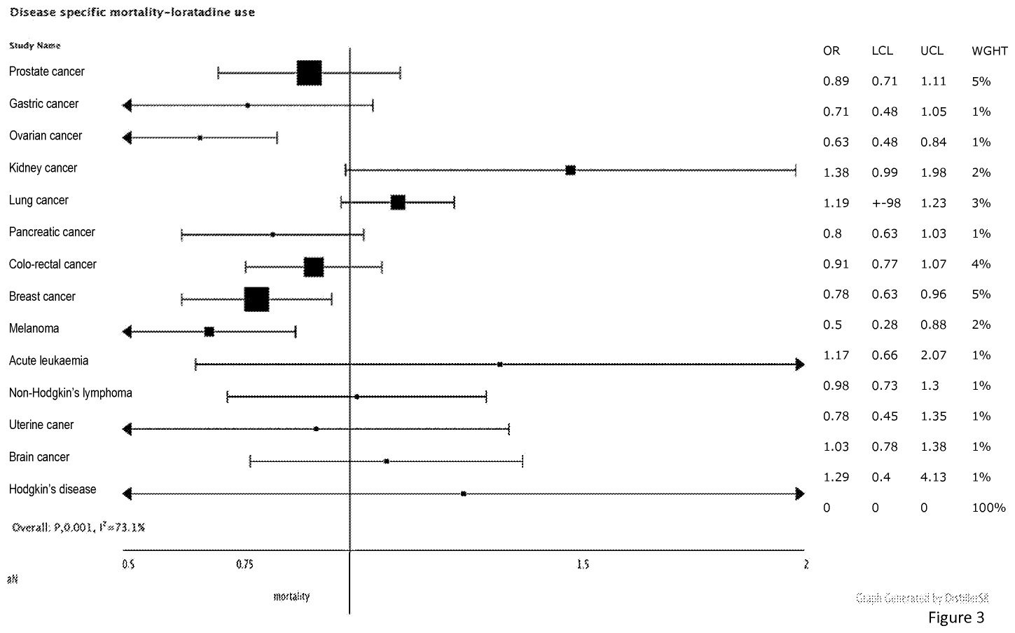

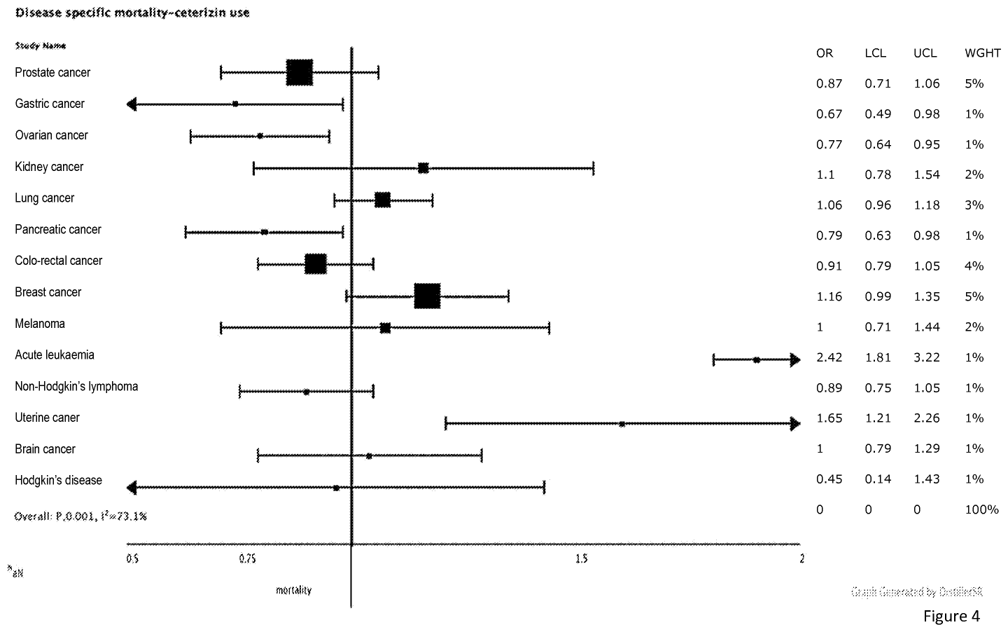

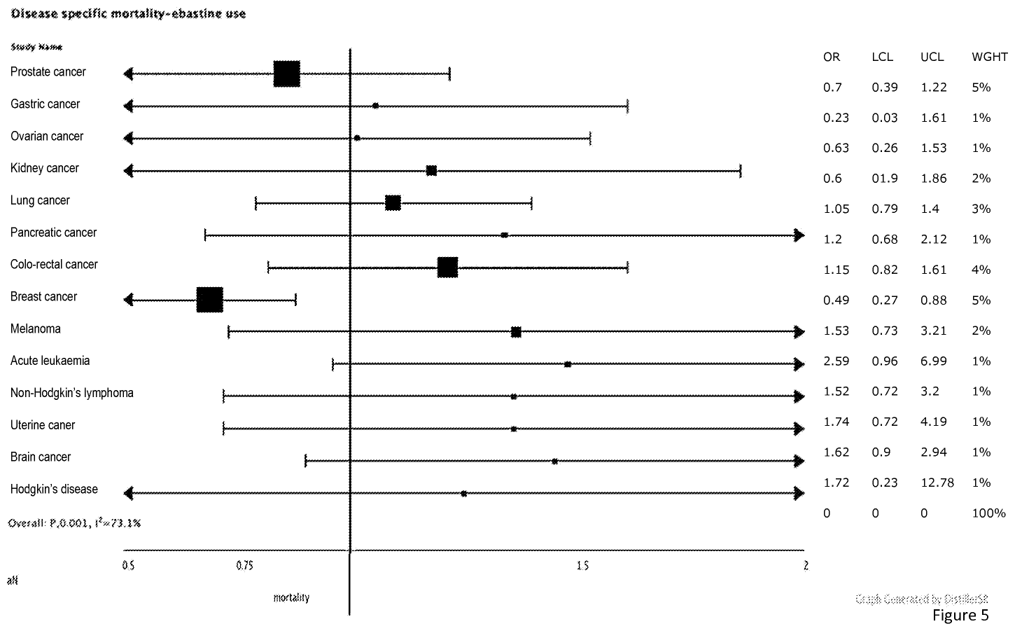

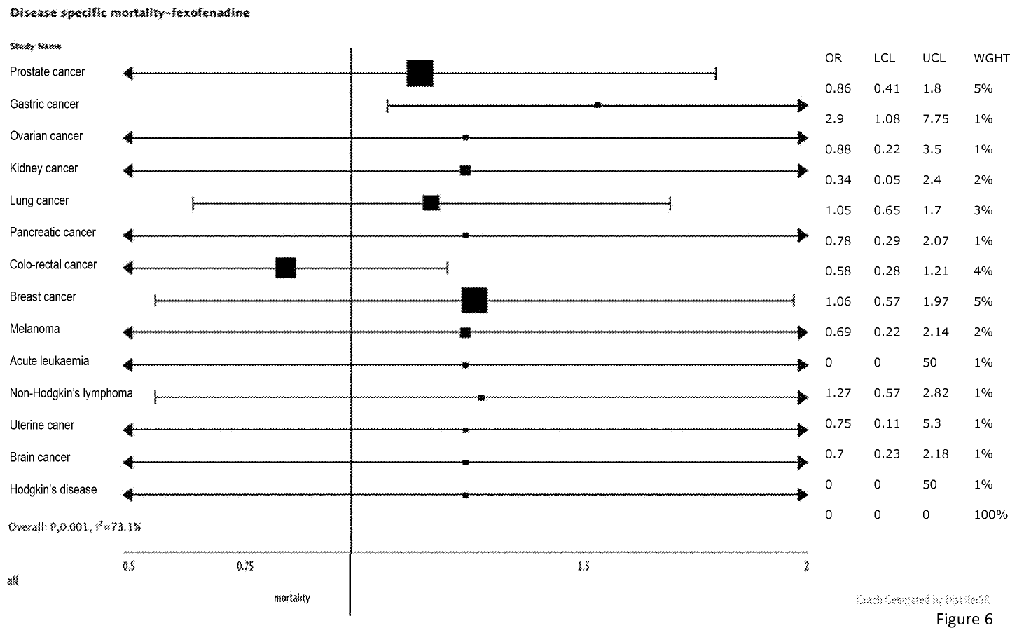

The results are robust, as the study was done on the entire Swedish population. The survival effects of the different antihistamines on different cancers are shown in FIGS. 1 to 6.

It was surprisingly found a strong survival benefit of desloratadine use in a diverse set of tumour types, namely lung cancer, prostate cancer, pancreatic cancer, gastric cancer, kidney cancer, ovarian cancer, colorectal cancer and Hodgins's disease. Whereas no effect were observed with other tumour types, e.g. brain cancer, uterine cancer and lympho-hematopoetic malignancies. In FIG. 1 is shown the survival benefit of desloratadine.

As can be seen in FIGS. 1 to 6, data is very diverse between antihistamines, which indicates that anti-histamines have substantial different biological effects among themselves although their main target is to block the histamine-1 receptor (H-1). Compared with other anti-histamines desloratadine has a bioavailability in all tissue blocking all receptors. However, other effects in the immune system have been described as effect on myeloid suppressor cells, dendritic cells and converting Th-1 response to Th-2 response. The effect seen in this study suggests that there is a possibility that other parts of the immune system could be affected.

Surprisingly, effect is seen a number of tumours that previously have responded to immune check blockade (checkpoint blockade immunotherapy). Checkpoint blockade immunotherapy has shown promise in treating a variety of malignancies, including; gastric cancer, kidney cancer, colo-rectal cancer, lung cancer, Hodgkin's disease, bladder cancer, liver cancer, melanoma, breast cancer and nasopharyngeal cancer.

Also, similarly to immune check blockade treatment, no or minor effects were seen for other types of cancers, e.g. brain cancer, uterine cancer and lympho-hematopoetic malignancies.

Survival curves becoming parallel to non-users suggest (in similarity with the check point blockade) a possibility of a curative therapy.

Since there is a very clear positive and negative overlap, it is suggested that cancers that respond to checkpoint blockade immunotherapy will respond to desloratadine treatment.

A possibility is that the findings coincide with the T-lymphocyte-associated protein 4 (CTLA-4)/programmed cell death protein 1 (PD-1) or PD-L1 pathways. This is supported by the effects among the different tumour types of which several are known to respond to immune checkpoint blockade. CTLA-4 is thought to regulate T-cell proliferation early in an immune response, primarily in lymph nodes, whereas PD-1 suppresses T cells later in an immune response, primarily in peripheral tissues, and these have been found to be reliable targets of immune cell activation of anti-tumor immune responses for the treatment of cancer.

In one embodiment desloratadine is thus provided for use in the treatment and/or prophylactic treatment and/or treatment of lung cancer, prostate cancer, pancreatic cancer, gastric cancer, kidney cancer, ovarian cancer, colorectal cancer, and Hodgins's disease, bladder cancer, liver cancer, and/or nasopharyngeal cancer. Further, desloratadine is provided for use in the prevention and/or treatment of other cancers known to respond to checkpoint blockade immunotherapy. The checkpoint blockade immunotheraphy may be immunotheraphy affecting and/or inhibiting the T-lymphocyte-associated protein 4 (CTLA-4)/programmed cell death protein 1 (PD-1) and/or PD-L1 pathways.

In one further embodiment, a pharmaceutical composition comprising desloratadine is provided for use in the treatment and/or prophylactic treatment of lung cancer, prostate cancer, pancreatic cancer, gastric cancer, kidney cancer, ovarian cancer colorectal cancer, and Hodgins's disease, bladder cancer, liver cancer, and/or nasopharyngeal cancer, wherein the composition comprises desloratadine. Further, such a composition is provided for use in the prevention and/or prophylactic treatment of other cancers known to respond to checkpoint blockade immunotherapy. The checkpoint blockade immunotherapy may be immunotherapy affecting and/or inhibiting the T-lymphocyte-associated protein 4 (CTLA-4)/programmed cell death protein 1 (PD-1) and/or PD-L1 pathways.

In one embodiment, the cancer to be treated is selected from the group consisting of lung cancer, prostate cancer, pancreatic cancer, gastric cancer, kidney cancer, ovarian cancer, colorectal cancer, and Hodgins's disease. In one embodiment, the cancer to be treated is selected from the group consisting of lung cancer, prostate cancer, pancreatic cancer, gastric cancer, colorectal cancer, and Hodgins's disease.

The non-responding tumours in the study includes tumours originating in the lympho-hematopoetic tissue or in the brain. This indicates that diseases affecting lymphatic effector cell tissue or the brain with a different lymphatic system are unresponsive.

In one embodiment, the cancers are cancers not related to lymphatic effector cell tissue or the brain.

As can be seen in table 1, several of these cancers are indeed very serious for a patient—a 5 year survival chance for a patient with gastric cancer was only 25% 2016 (table 1), and this chance is increased by 30% if desloratadine is used for treatment of said cancer (FIG. 1). In one embodiment, desloratadine is for use in the treatment and/or prophylactic treatment of gastric cancer.

Similarly, a 5 year survival chance for a patient with colorectal cancer was only about 65% 2016 (table 1), and this chance is increased by 15% if desloratadine is used for treatment of said cancer (FIG. 1). In one embodiment, desloratadine is for use in the treatment and/or prophylactic treatment of colo-rectal cancer.

Similarly, a 5 year survival chance for a patient with pancreatic cancer was only about 8% 2016 (table 1), and this chance is increased by 35% if desloratadine is used for treatment of said cancer (FIG. 1). This is extremely welcome news, since advanced pancreatic cancer is often inoperable. In one embodiment, desloratadine is for use in the treatment and/or prophylactic treatment of pancreatic cancer.

Similarly, a 5 year survival chance for a patient with lung cancer was only about 20% 2016 (table 1), and this chance is increased by 15% if desloratadine is used for treatment of said cancer (FIG. 1). In one embodiment, desloratadine is for use in the treatment and/or prophylactic treatment of lung cancer.

Similarly, a 5 year survival chance for a patient with kidney cancer was only about 75% 2016 (table 1), and this chance is increased by 30% if desloratadine is used for treatment of said cancer (FIG. 1). In one embodiment, desloratadine is for use in the treatment and/or prophylactic treatment of kidney cancer.

Similarly, a 5 year survival chance for a patient with ovarian cancer was only about 55% 2016 (table 1), and this chance is increased by 17% if desloratadine is used for treatment of said cancer (FIG. 1). In one embodiment, desloratadine is for use in the treatment and/or prophylactic treatment of ovarian cancer.

Similarly, a 5 year survival chance for a patient with Hodgkin's disease was only about 86% 2016 (table 1), and this chance is increased by 17% if desloratadine is used for treatment of said cancer (FIG. 1). In one embodiment, desloratadine is for use in the treatment and/or prophylactic treatment of Hodgkin's disease.

In one embodiment, the cancer is one, two, three, four, five, or six cancers selected from the group consisting of lung cancer, prostate cancer, pancreatic cancer, gastric cancer, kidney cancer, ovarian cancer, colorectal cancer and Hodgins's disease.

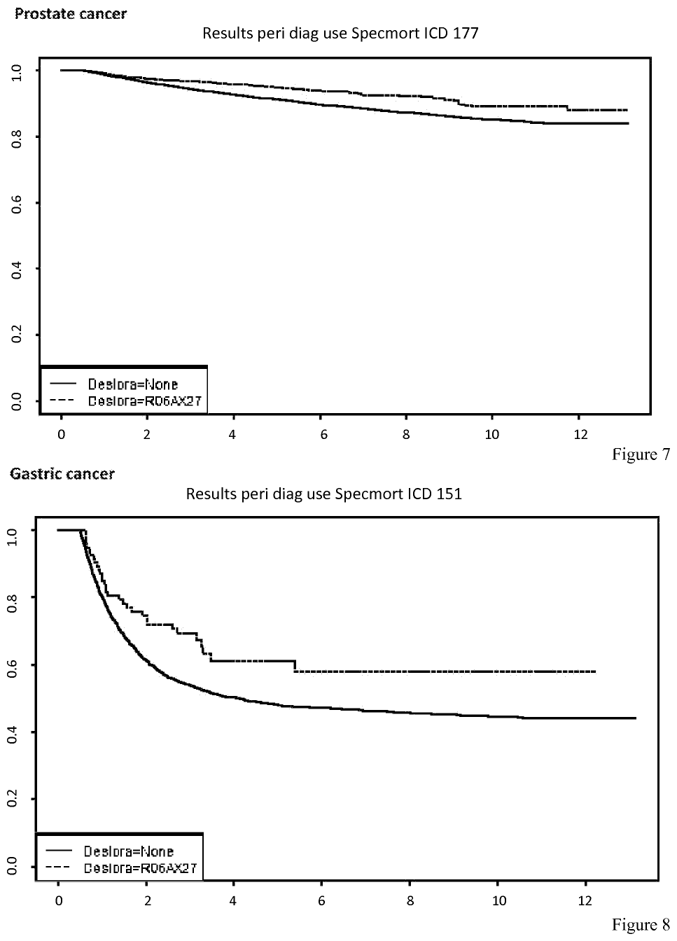

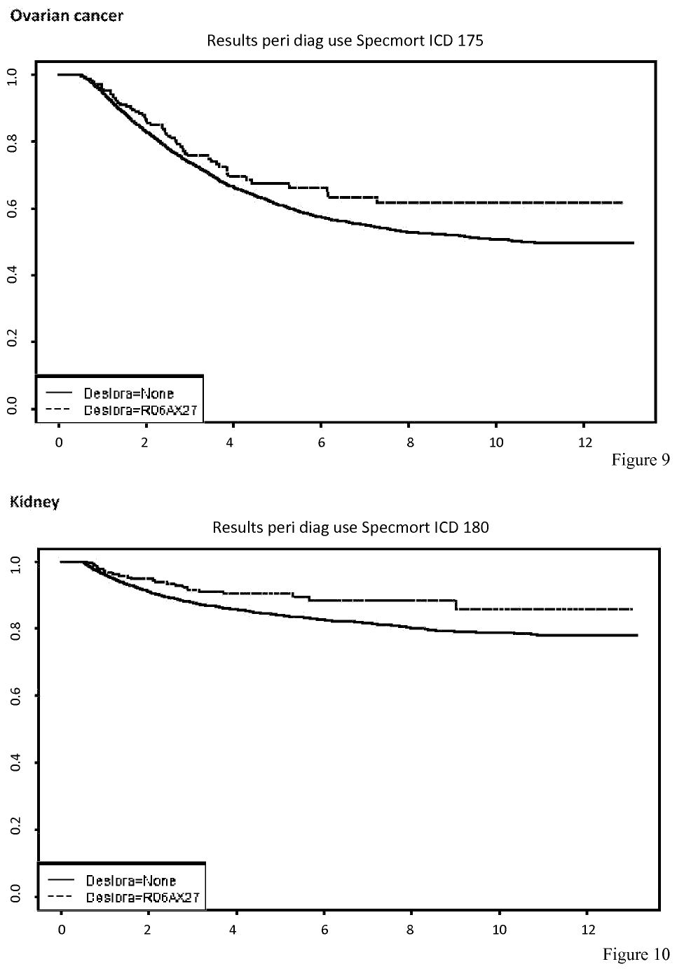

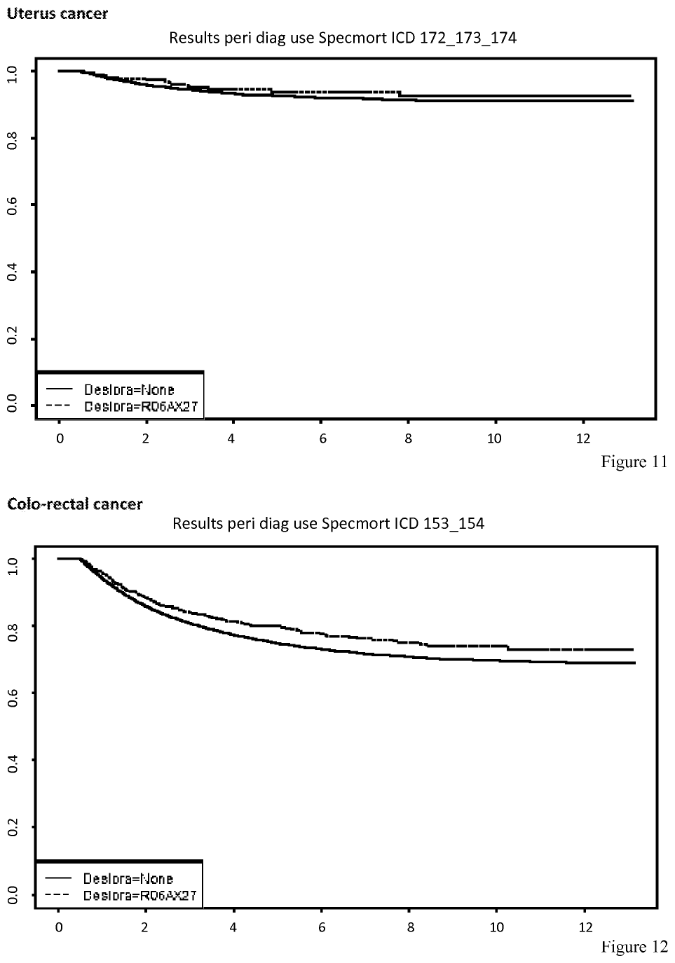

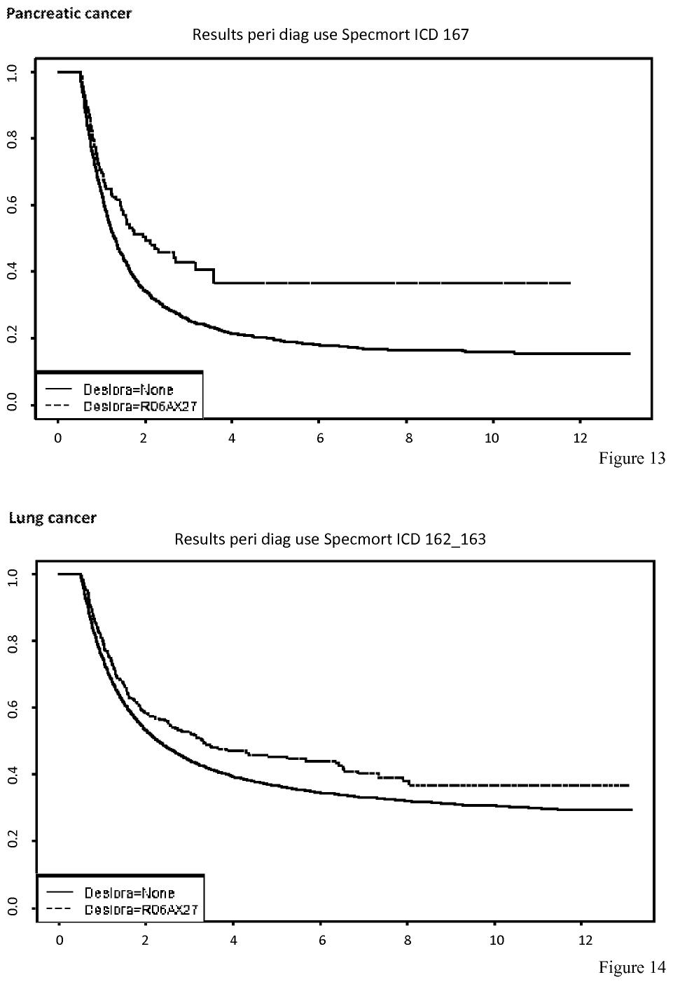

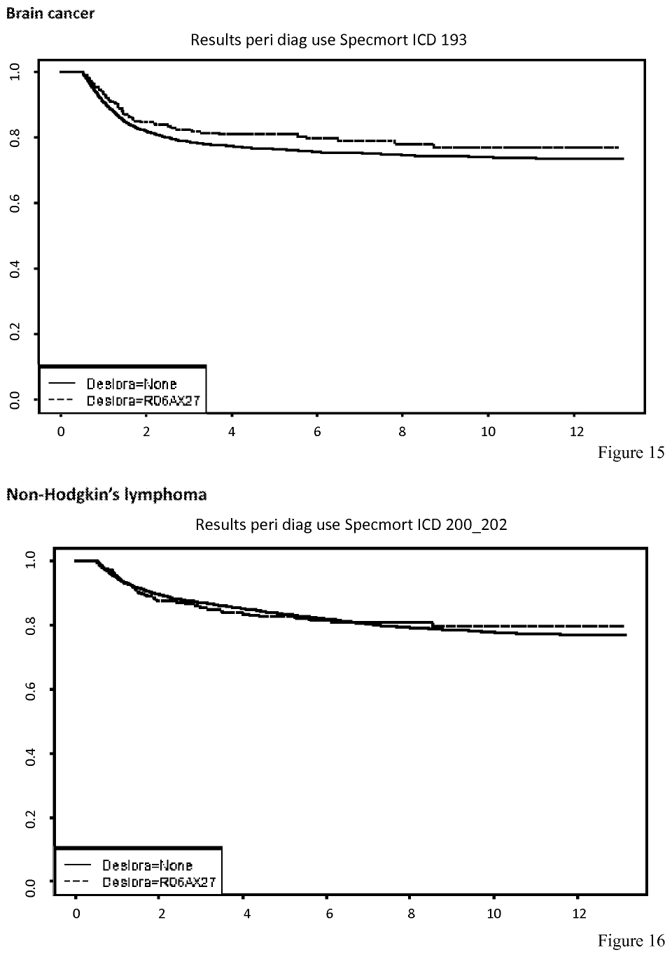

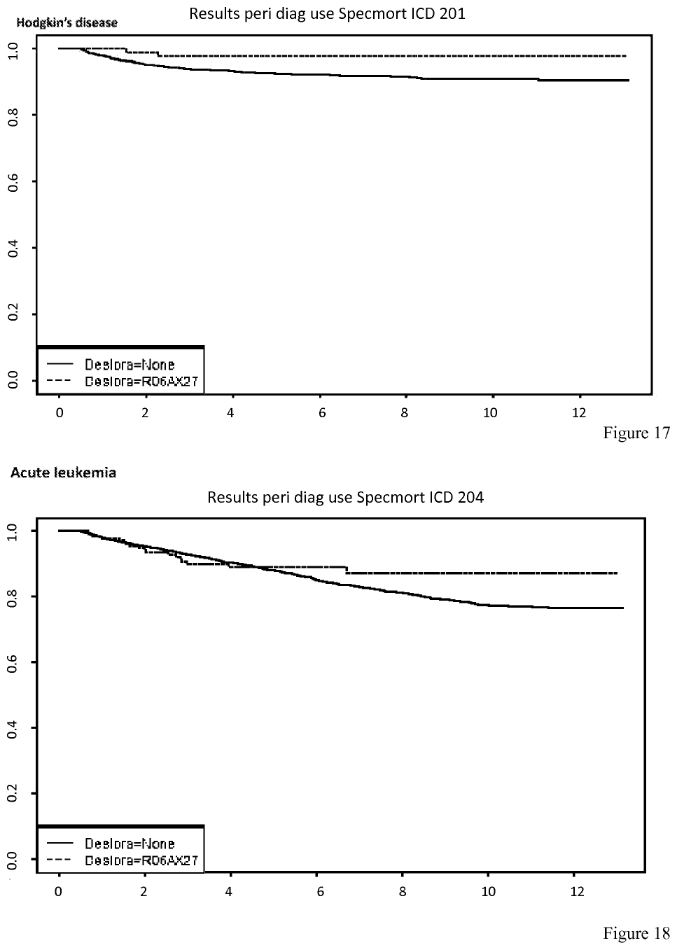

Importantly, survival curves, with follow up parallel to non-treated, suggest curative effect for 10-20% for desloratadine use for said cancers. Survival curves for these cancers can be seen as FIGS. 7 to 18. Most cancers that are going to come back will do so in the first 2 years or so after treatment. After 5 years, you are even less likely to get a recurrence. For some types of cancer, after 10 years your doctor may say that you are cured. In the survival curves, the curve for the non-treated patients gradually becomes parallel to the curve for the patients treated with desloratadine, which shows that the effect is permanent (i.e. the patients are essentially cured) for the duration of the test (>12 years).

In one embodiment, the treatment has a curative effect of up to 30% for a patient diagnosed with said cancers, such as a curative effect of from 10 to 20% for patients diagnosed with said cancers.

Excitingly, for some of the affected cancers, it is the first time that a non-surgical therapy so substantially improves survival rate, for instance for pancreatic and gastric cancer. For pancreatic cancer, having a 5 year survival of only around 8% (table 1), the data with desloratadine is astonishing and indicates the possibility of a curative effect for a proportion of patients.

In one embodiment, the treatment is a non-surgical therapy for pancreatic or gastric cancer.

Desloratadine is a second-generation H1-antihistamine. Second-generation H1-antihistamines are newer drugs that are more selective for peripheral H1 receptors as opposed to the central nervous system H1 receptors and cholinergic receptors. They are very polar compounds, which mean that they do not cross the blood-brain-barrier (BBB) and will act mainly outside the central nervous system. Also, desloratadine has minimal side effects. It remains continuously used and is prescribed to adults and children alike, and is widely available without a prescription, due to its safety. The fact that desloratadine is safe and commonplace drugs make it an excellent candidate for repurposing purposes. Also, since side effects are rare, prophylactic treatment is possible for patients with high risk factors for developing a new cancer or having been treated for cancer, with high risk for having a recurrence of cancer.

According to an embodiment, the use of in desloratadine treatment and/or prophylactic treatment is for patients with high risk factors for or developing a new cancer, or having been treated for cancer, with high risk for having a recurrence of cancer.

In one embodiment, the patient has previously been diagnosed and treated for said cancer(s) and the desloratadine treatment reduces recurrence of said cancer.

In one embodiment, the treatment is peridiagnostic, such as from at least 100 days, such at least 3 months, such as at least 6 months, such as at least 1 year before, and such as from at least 100 days, such at least 3 months, such as at least 6 months, such as at least 1 year, such as at least 2 years after cancer diagnosis.

The data indicates that longer therapy is better than shorter (indicated in FIGS. 7 to 18). Also, the risk for and side effects from desloratadine use is very low, even during prolonged use. A prolonged treatment is thus desirable.

In one embodiment, the treatment period is at least 50 days, such as at least 100 days, such as at least 200 days, such as at least 1 year, such as at least 2 years, such as at least 3 years, such as at least 5 years.

In this study, the drug usage is estimated based on drug prescriptions. This information reflects usage fairly accurate, especially for persons with multiple prescriptions. A confounding factor is the possibility of off the counter purchase of cetirizine and loratadine, though this should only dilute any seen effects. Desloratadine and clemastine however, were both prescription drugs, during the time of this study.

Thus, in an embodiment of the present invention, the daily dose of desloratadine corresponds to the defined daily dose (DDD).

The DDD is a statistical measure of drug consumption, defined by the World Health Organization (WHO).

Five milligram of desloratadine is the commonly prescribed daily dose in the prescription register and very few patients have been given a higher daily dose. As such, this study shows a clear benefit from antihistamine treatment in normal dose regimes, but other dose regimes are equally possible, for instance a higher dose for perioperational treatment.

Thus, in one embodiment of the present invention, the dose of the desloratadine is between 2.5 to 45 mg per day, preferably between 5 to 20 mg per day, most preferably 5 mg per day.

Multi dose studies with 45 mg per day of desloratadine did not show any further clinical side effects (FASS, Swedish Medicines Compendium for healthcare professionals), showing he possibility for high doses of desloratadine.

Thus, in one embodiment, the dose of the desloratadine is between 45 to 250 mg per day, preferably between 85 to 150 mg per day most preferably 100 mg per day.

The majority of the patients in the study have received some kind of cancer treatment, such as surgical treatment, chemotherapy or radiochemotherapy treatment. As such, the study demonstrates that the desloratadine treatment can be a co-treatment with other cancer treatments. This is most likely preferable for cancer forms where other treatments, such as surgical treatment, is possible and have a high success rate. In such cases, such treatments are often combined with other treatments to minimize the risk of recurrence of the cancer. Here, desloratadine presents a very desirable option, due to having very few side effects and a high likelihood that the patients will be able to take the medication.

In one embodiment, the treatment is a co-treatment together with at least one additional cancer treatment. In one further embodiment, said additional cancer treatment is at least one treatment selected from radiotherapy, chemotherapy, hormonal treatment and immunotherapy. In one further embodiment, additional cancer treatment is immunotherapy.

Unfortunately, despite promising results in some indications, only a subset of patients initially responds to PD-1 and CTLA-4 checkpoint inhibitors. Immune check point inhibitors (ICI) produce durable responses in about 20-40% of patients. Clinical evidence reveals that a substantial percentage of initial responders will ultimately relapse with lethal, drug-resistant disease months or years after therapy. To render ICIs more effective for a larger fraction of patients, combination approaches are of interest. The hypothesis is that the combinatorial effect will elicit a strong synergistic effect, which will be more potent than the sum of its parts—i.e. monotherapies.

As such, in one embodiment, said co-treatment is treatment with immune check point inhibitors in combination with desloratadine.

Examples of such inhibitors are PD-1 inhibitors nivolumab, pembrolizumab and cemiplimab, PD-L1 inhibitors atezolizumab, avelumab and durvalumab, or ipilimumab which targets CTLA-4.

The improvement seen in FIGS. 1 and 7 to 18 includes improved prognosis as well as increase in survival rate.

Thus, in one embodiment, the treatment seeks to improve the prognosis for a patient diagnosed said cancers. In one embodiment, the treatment seeks to increase the survival time for a patient diagnosed with said cancers.

In the context of the present specification, the term “therapy” and “treatment” includes prevention or prophylaxis, unless there are specific indications to the contrary. The terms “therapeutic” and “therapeutically” should be construed accordingly.

According to an embodiment, treatment does also encompass pre-treatment, i.e. prophylactic treatment.

According to an embodiment, the term “prophylaxis” or “prophylactic” includes primary prophylaxis, secondary prophylaxis, tertiary prophylaxis or periodic prophylaxis, unless there are specific indications to the contrary. Primary prophylaxis refers to the preventive treatment of an initial disease. Secondary prophylaxis refers to reducing the incidence of recurrence or reactivation of a pre-existing disease. Teritary prophylaxis refers to continuous treatment started after the onset of a disease to mitigate further damage. Periodic prophylaxis refers to periodic prophylactic treatment given for shorter periods of time. For instance, a H1 receptor antihistamine being desloratadine or loratadine used in perioperational treatment may be used a secondary prophylaxis for reducing the incidence of recurrence or reactivation of a pre-existing cancer.

Thus, in one embodiment of the present invention, treatment does relate to primary prophylaxis, secondary prophylaxis, tertiary prophylaxis or periodic prophylaxis.

Desloratadine may obviously also be used in method for treating such diseases and disorders as have been disclosed herein e.g. treatment of lung cancer, prostate cancer, pancreatic cancer, gastric cancer, kidney cancer, ovarian cancer, colorectal cancer, Hodgins's disease, bladder cancer, liver cancer, and/or nasopharyngeal cancer. Such a method includes the step of administering an effective amount of desloratadine to a subject in need for treatment of lung cancer, prostate cancer, pancreatic cancer, gastric cancer, kidney cancer, ovarian cancer, colorectal cancer, Hodgins's disease, bladder cancer, liver cancer, and/or nasopharyngeal cancer.

Evidently, desloratadine may also be used for the manufacture of a medicament for use in such treatment as disclosed herein, e.g. treatment and/or prophylactic treatment of lung cancer, prostate cancer, pancreatic cancer, gastric cancer, kidney cancer, ovarian cancer, colorectal cancer, Hodgins's disease, bladder cancer, liver cancer, and/or nasopharyngeal cancer.

Thus, one embodiment relates to a pharmaceutical composition, comprising desloratadine and an excipient, for use in the treatment and/or prophylactic treatment of lung cancer, prostate cancer, pancreatic cancer, gastric cancer, kidney cancer, ovarian cancer, colorectal cancer, Hodgins's disease, bladder cancer, liver cancer, and/or nasopharyngeal cancer.

An excipient is a natural or synthetic substance formulated alongside the active ingredient of a medication. Purpose includes stabilization, bulking, facilitating drug absorption, reducing viscosity, or enhancing solubility. Excipients may also be useful for the manufacturing process, to aid in the handling of the active substance. The selection of appropriate excipients also depends upon the route of administration and the dosage form, as well as the active ingredient and other factors. In one aspect of the invention, the pharmaceutical composition comprises an excipient selected from the list containing lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, methyl cellulose, polyethylene glycol, poloxamers, lactose, hydroxypropylmethylcellulose (HPMC), hydroxypropyl cellulose (HPC), lactose, and polyvinylpyrrolidone.

Material and methods Linking the Cancer Registry with the Drug Prescription Registry and Cause of Death Registry the survival of over 500 000 cancer patients with cancer in the breast-, prostate, ventrikel, pancreas, kidney, colo-rectal, lung, uterus, ovary, brain, melanoma, lymph gland and bone marrow was studied between 2005-2014. The survival was studied for main users of desloratadine, loratadine, ebastine, cetirizine, fexofenadine and clemastine.

Use of the six major antihistamines was established using the Swedish Prescribed Drug Register, a record of all dispensed prescribed pharmaceuticals in Sweden since July 1st 2005. To be included in the registry at least 2 weeks of prescription is required. Prescription free antihistamine use, non-dispensed doses, or use of other, less common, types of antihistamines is here considered non-use. Causes of death were obtained from the Swedish Cause of Death Register, a database where all deaths have been recorded since 1952. Data was pseudo-anonymized and the study was approved by the Regional Ethics Board.

We analyzed both the peri- and cumulative post-diagnostic use of antihistamines among melanoma patients. Peri-diagnostic use was a proxy for ever-use, and is here defined as use within six months pre-diagnosis and six months post-diagnosis, where the main antihistamine (in terms of dispensed defined daily doses, or DDDs) used by each individual during this time was identified. The analysis was done using a Cox regression model with time to even or censoring in years as a time scale. Subgroup analyses were made for groups based on age and gender (as a proxy for sex).

For the cumulative post-diagnostic antihistamine use analysis, mortality rates for antihistamine users were compared to those of non-users through a Cox regression model allowing for time varying covariates, and survival time was measured in years since diagnosis. This allowed for a single individual to provide data as both a non-user and user of different antihistamines. The impact of the cumulative DDDs was assessed after log transformation of the DDDs plus one3. Cox regression analyses were stratified for patient age at diagnosis, tumor size and node status (T and N status, respectively). A lag-time analysis was performed as described by Tamim et al. and Richardson et al. Statistical analyses were performed using R version XXX6. Two-tailed P-values were used, and a test based on Schoenfeld residuals was done to assess the proportional hazards assumption, and was accepted, both for disease-specific and overall mortality.

First the study uses prescriptions and there is a possibility that the individual partly has not taken the medicine, 2012-2014 patent expired for different antihistamines and therefore opportunistic use may partly diminish the effect. We have assessed the main type of anti-histamines used and a minor group has used more than one type (<10%). Information on stage, socio-economic factors, tumour therapies, and prognostic tumour factors are not present in the national registries. As the autopsy rate is low in Sweden <5% disease specific mortality may be underestimated.

Results

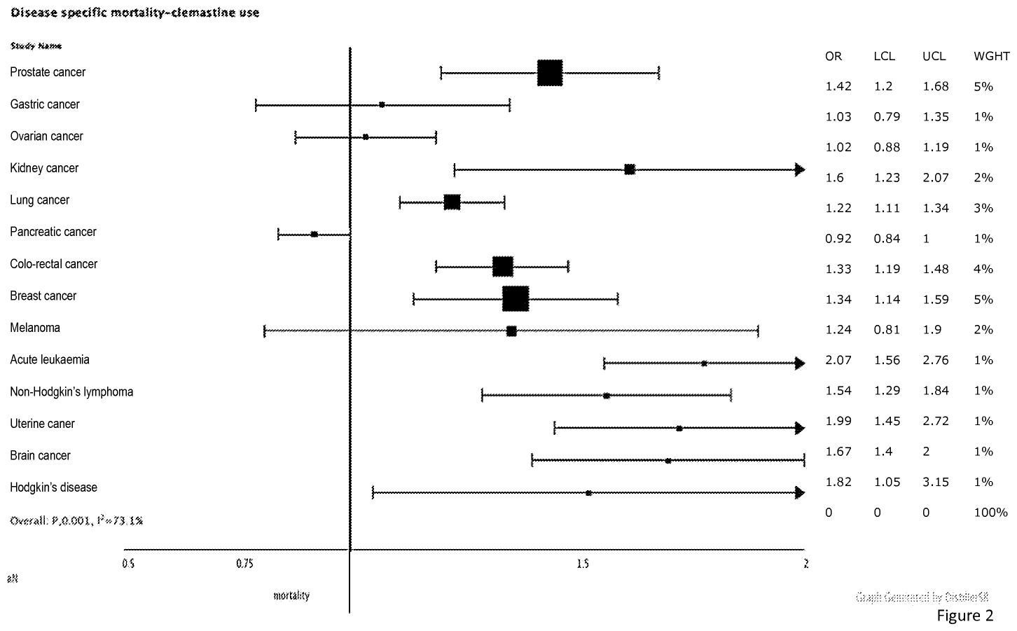

Below are summarized the results from the study. We found a consistently beneficial effect of desloratadine use on lung cancer, prostate cancer, pancreatic cancer, gastric cancer, kidney cancer, ovarian cancer and colorectal cancer survival (and breast cancer, as previously shown), but not for uterine-, and brain cancer and lympho-hematopoetic diseases, regardless of age, gender and tumor status. This is plotted as a forest plot in FIG. 1. Also shown are forest plots for the other H1-antihistamines (cetirizine, clemastine, ebastine, fexofenadine and loratadine) in FIGS. 2-6.

In users of loratadine survival was better in ovarian cancer. Ceterizine users had a better survival in pancreatic-, gastric- and ovarian cancer. Ebastine use increased breast cancer survival, while fexofenadine for all tumour types was insignificant. Clemastine users consistently showed a worse survival.

Survival curves are provided as FIGS. 7 to 18. For Loratadine, most survival curves became parallel to non-users suggesting a long time curative effect.

The figures shows the cancers and the corresponding hazard ratios (HR). A hazard ratio below 1.0 indicates better survival, while as hazard ratio above 1.0 indicates worse survival, when compared to non-users. The confidence interval (95% CI) indicates the level of uncertainty around the measure of effect. A 95% confidence interval means that the confidence interval covers the true value in 95 of 100 studies performed. The p-value is a probability, where a small p-values correspond to strong evidence. P<0.05 indicates a statistically significant difference between groups.

Although the present invention has been described above with reference to (a) specific embodiment(s), it is not intended to be limited to the specific form set forth herein. Rather, the invention is limited only by the accompanying claims and, other embodiments than the specific above are equally possible within the scope of these appended claims, e.g. different than those described above.

In the claims, the term “comprises/comprising” does not exclude the presence of other elements or steps. Furthermore, although individually listed, a plurality of means, elements or method steps may be implemented by e.g. a single unit or processor. Additionally, although individual features may be included in different claims, these may possibly advantageously be combined, and the inclusion in different claims does not imply that a combination of features is not feasible and/or advantageous. In addition, singular references do not exclude a plurality. The terms “a”, “an”, “first”, “second” etc do not preclude a plurality. Reference signs in the claims are provided merely as a clarifying example and shall not be construed as limiting the scope of the claims in any way.

1. Desloratadine for use in the treatment and/or prophylactic treatment of lung cancer, prostate cancer, pancreatic cancer, gastric cancer, kidney cancer, ovarian cancer, colorectal cancer, Hodgins's disease, bladder cancer, liver cancer, and/or nasopharyngeal cancer.

2. Desloratadine for use according to claim 1, wherein the cancer is one or several selected from the group consisting of lung cancer, prostate cancer, pancreatic cancer, gastric cancer, kidney cancer, ovarian cancer, colorectal cancer and Hodgins's disease.

3. Desloratadine for use according to claim 1, wherein the cancer is one or several selected from the group consisting of lung cancer, prostate cancer, pancreatic cancer, gastric cancer, colorectal cancer and Hodgins's disease.

4. Desloratadine for use according to claim 1, wherein the daily dose of desloratadine corresponds to the defined daily dose (DDD).

5. Desloratadine for use according to claim 1, wherein the daily dose of desloratadine is from 2.5 to 45 mg per day, preferably between 5 to 20 mg per day, more preferably about 5 mg per day.

6. Desloratadine for use according to claim 1, wherein a treatment period is at least 50 days, such as at least 100 days, such as at least 200 days, such as at least 1 year, such as at least 3 years.

7. Desloratadine for use according to claim 1, wherein the patient has previously been diagnosed and treated for said cancer(s) and the use of desloratadine treatment reduces recurrence of said cancer.

8. Desloratadine for use according to claim 1, wherein the treatment and/or prophylactic treatment is a co-treatment together with at least one additional cancer treatment.

9. Desloratadine for use according to claim 8, wherein said additional cancer treatment is at least one treatment selected from radiotherapy, chemotherapy, hormonal treatment and immunotherapy.

10. Desloratadine for use according to claim 8, wherein said co-treatment is treatment with immune check point inhibitors in combination with desloratadine.
